(19) United States
(12) Patent Application Publication
(10) Pub. No.: US 2023/0200666 A1
(43) Pub. Date: Jun. 29, 2023

(54) ARTERIAL-PRESSURE ESTIMATION APPARATUS, ARTERIAL-PRESSURE ESTIMATION SYSTEM, AND ARTERIAL-PRESSURE ESTIMATION METHOD

(71) Applicant: TERUMO KABUSHIKI KAISHA, Shibuya-ku (JP)

(72) Inventors: Tomoki UTSUGIDA, Machida-shi (JP); Tomonori HATTA, Hadano-shi (JP); Yoshiharu EZASHI, Ashigarakamigun Nakaimachi (JP)

(73) Assignee: TERUNO KABUSHIKI KAISHA, Shibuya-ku (JP)

(21) Appl. No.: 18/172,395

(22) Filed: Feb. 22, 2023

Related U.S. Application Data
(63) Continuation of application No. PCT/JP2021/033604, filed on Sep. 13, 2021.

(30) Foreign Application Priority Data
Sep. 15, 2020 (JP) 2020-154967

(51) Int. Cl. A61B 5/021; A61B 5/00; A61B 5/022

(52) U.S. Cl. CPC A61B 5/02116; A61B 5/7278; A61B 5/02233; A61B 5/7267; A61B 2562/0247

(57) ABSTRACT

An arterial-pressure estimation apparatus includes a control unit configured to acquire sensor data indicating timings at which blood flows generated, for one heartbeat, at two or more locations of a blood vessel downstream of an aorta when the at two or more locations are compressed such that a location of the two or more locations that is further downstream of the aorta has a higher compression pressure than a location of the two more locations that is further upstream of the aorta, and estimate a temporal variation of an arterial pressure based on the acquired sensor data.

### CROSS-REFERENCES TO RELATED APPLICATIONS

This application is a continuation of International Application No. PCT/JP2021/033604 filed on Sep. 13, 2021, which claims priority to Japanese Application No. 2020-154967 filed on Sep. 15, 2020, the entire content of both of which is incorporated herein by reference.

### TECHNOLOGICAL FIELD

The present disclosure generally relates to an arterial-pressure estimation apparatus, an arterial-pressure estimation system, and an arterial-pressure estimation method.

### BACKGROUND DISCUSSION

U.S. Patent Application Publication No. 2015/0265163 A discloses a technique for estimating a left intraventricular pressure from heart sounds, upper-arm cuff pressures, and Korotkoff sounds (K sounds).

In a medical examination and treatment for heart failure, a left intraventricular pressure of a heart is an important observation item. However, when the left intraventricular pressure is directly measured, a device, such as a sensor, can be put in the heart, and which invasiveness to the patient is relatively high.

In the technique disclosed in U.S. Patent Application Publication No. 2015/0265163 A, data is collected over a plurality of arterial-pressure waveforms, and thus an arterial-pressure waveform as a correct value varies. Specifically, in a process of loosening a tightening pressure of a cuff (i.e., an inflatable cuff that wraps around the arm of a patient), waveforms corresponding to a plurality of heartbeats are identified, and information collected from the waveforms is integrated to estimate an arterial-pressure waveform. Therefore, the estimation precision is relatively low.

### SUMMARY

The present disclosure relates to non-invasively estimating a temporal variation of an arterial pressure with relatively high precision.

An arterial-pressure estimation apparatus according to an aspect of the present disclosure includes a control unit configured to acquire sensor data indicating timings at which blood flows generated, for one heartbeat, at two or more locations of a blood vessel downstream of an aorta when the two or more locations are compressed such that a location of the two or more locations that is further downstream of the aorta has a higher compression pressure than a location of the two or more locations that is further upstream of the aorta, and estimate a temporal variation of an arterial pressure based on the acquired sensor data.

As an embodiment, the control unit may be configured to correct, according to a distance between the two or more locations, the timings indicated by the sensor data, and estimate, according to the corrected timings, and pressures by which the two or more locations are compressed, a temporal variation of the arterial pressure.

As an embodiment, the control unit may be configured to correct the timings by subtracting a delay time corresponding to the distance, from the timings indicated by the sensor data.

As an embodiment, the control unit may be configured to estimate a left ventricular end-diastolic pressure (LVEDP) on the basis of an estimation result of the temporal variation of the arterial pressure.

As an embodiment, the control unit may be configured to estimate, as the temporal variation of the arterial pressure, an arterial-pressure waveform, estimate, according to the estimated arterial-pressure waveform, a left-ventricular-pressure waveform, and acquire, from the estimated left-ventricular-pressure waveform, an estimated value of the LVEDP.

As an embodiment, the control unit may be configured to input the estimation result of the temporal variation of the arterial pressure, into a trained model, and acquire an estimated value of the LVEDP from the trained model.

As an embodiment, the control unit may be configured to output an estimated value of the LVEDP.

As an embodiment, the control unit may be configured to acquire the sensor data for a plurality of heartbeats, execute statistical processing on the sensor data, and estimate, on the basis of a result of the statistical processing, a temporal variation of the arterial pressure.

As an embodiment, the two or more locations may be three locations.

An arterial-pressure estimation system according to an aspect of the present disclosure includes: an arterial-pressure estimation apparatus that includes a control unit configured to acquire sensor data indicating timings at which blood flows generated, for one heartbeat, at two or more locations of a blood vessel downstream of an aorta when the two or more locations are compressed such that a location of the two or more locations that is further downstream of the aorta has a higher compression pressure than a location of the two more locations that is further upstream of the aorta, and estimate a temporal variation of an arterial pressure based on the acquired sensor data; and two or more sensors corresponding to the two or more locations on a one-to-one basis, and configured to detect blood flows for the one heartbeat generated at the corresponding locations, respectively.

As an embodiment, the arterial-pressure estimation system may further include two or more inflatable units corresponding to the two or more locations on a one-to-one basis, and configured to compress the corresponding locations, respectively.

As an embodiment, the two or more inflatable units may each be a cuff.

As an embodiment, the two or more inflatable units may each be an airbag, and the two or more airbags may be contained in a common cuff.

An arterial-pressure estimation method according to an aspect of the present disclosure includes: compressing two or more locations of a blood vessel downstream of an aorta with two or more inflatable units, and wherein a location of the two or more locations that is further downstream of the aorta has a higher compression pressure than a location of the two or more locations that is further upstream of the aorta; detecting blood flows generated, for one heartbeat, at the two or more locations with two or more sensors; acquiring sensor data indicating timings at which the blood flows for the one heartbeat are detected when the two or more locations are compressed; and estimating a temporal variation of an arterial pressure based on the acquired sensor data.

According to the present disclosure, a temporal variation of an arterial pressure is non-invasively estimated with relatively high precision.

### BRIEF DESCRIPTION OF THE DRAWINGS

FIG. 1 is a block diagram illustrating a configuration of an arterial-pressure estimation system according to an embodiment of the present disclosure.

FIG. 2 is a block diagram illustrating a configuration of an arterial-pressure estimation apparatus according to the embodiment of the present disclosure.

FIG. 3 is a flowchart illustrating operations of the arterial-pressure estimation system according to the embodiment of the present disclosure.

FIG. 4A is a graph illustrating an example of an electrocardiographic waveform according to the embodiment of the present disclosure.

FIG. 4B is a graph illustrating an example of a first K-sound waveform according to the embodiment of the present disclosure.

FIG. 4C is a graph illustrating an example of a second K-sound waveform according to the embodiment of the present disclosure.

FIG. 4D is a graph illustrating an example of a third K-sound waveform according to the embodiment of the present disclosure.

FIG. 5 is a graph illustrating an example of points plotted in the embodiment of the present disclosure.

FIG. 6 is a graph illustrating an example of a curve estimated in the embodiment of the present disclosure.

FIG. 7 is a graph illustrating an example of curves extracted in the embodiment of the present disclosure.

FIG. 8 is a graph illustrating an example of a curve averaged in the embodiment of the present disclosure.

FIG. 9 is a graph illustrating an example of a left ventricular end-diastolic pressure (LVEDP) estimated in the embodiment of the present disclosure.

FIG. 10A is a diagram illustrating waveforms of an arterial pressure near an aortic valve, a left ventricular pressure, and heart sounds according to a comparative example.

FIG. 10B is a diagram illustrating a flow of blood corresponding to FIG. 10A.

FIG. 11A is a diagram illustrating the arterial-pressure waveform near the aortic valve, the left-ventricular-pressure waveform, the heart sound waveform, and an arterial-pressure waveform of an upper arm according to the comparative example.

FIG. 11B is a diagram illustrating a flow of blood corresponding to FIG. 11A.

FIG. 12A is a diagram illustrating the arterial-pressure waveform near the aortic valve, the left-ventricular-pressure waveform, the heart sound waveform, and the arterial-pressure waveform of the upper arm according to the comparative example.

FIG. 12B is a diagram illustrating a flow of blood corresponding to FIG. 12A.

FIG. 13A is a diagram illustrating the arterial-pressure waveform near the aortic valve, the left-ventricular-pressure waveform, the heart sound waveform, the arterial-pressure waveform of the upper arm, an electrocardiographic waveform, and a first K-sound waveform of the upper arm according to the comparative example.

FIG. 13B is a diagram illustrating a flow of blood corresponding to FIG. 13A.

FIG. 14 is a diagram illustrating the arterial-pressure waveform near the aortic valve, the left-ventricular-pressure waveform, the heart sound waveform, the arterial-pressure waveform of the upper arm, the electrocardiographic waveform, and a first K-sound waveform of the upper arm according to the comparative example.

FIG. 15 is a diagram illustrating the arterial-pressure waveform near the aortic valve, the left-ventricular-pressure waveform, the heart sound waveform, the arterial-pressure waveform of the upper arm, the electrocardiographic waveform, a first K-sound waveform of the upper arm, an estimated arterial-pressure waveform, and a fitting curve according to the comparative example.

FIG. 16 is a diagram illustrating the arterial-pressure waveform near the aortic valve, the left-ventricular-pressure waveform, the heart sound waveform, the arterial-pressure waveform of the upper arm, the electrocardiographic waveform, the first K-sound waveform of the upper arm, the estimated arterial-pressure waveform, the fitting curve, and an estimated LVEDP according to the comparative example.

### DETAILED DESCRIPTION

Hereinafter, as a comparative example, a method for estimating a left intraventricular pressure from an arterial-pressure waveform, similarly to the technique disclosed in U.S. Patent Application Publication No. 2015/0265163 A, will be described with reference to the drawings.

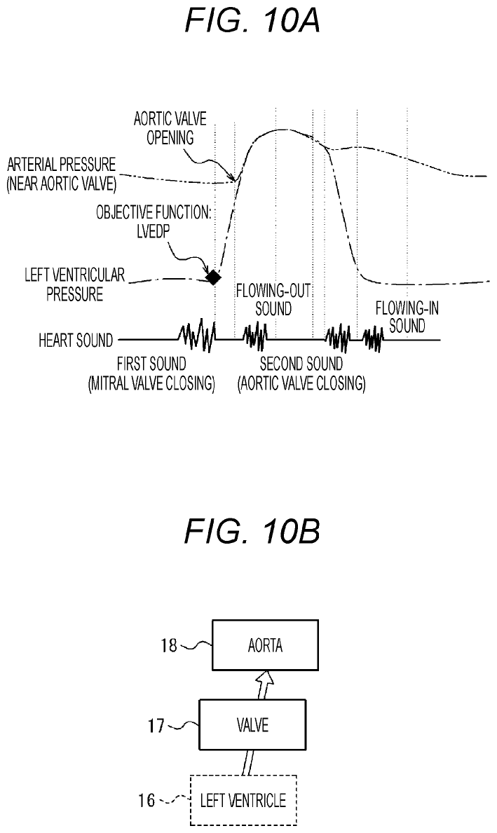

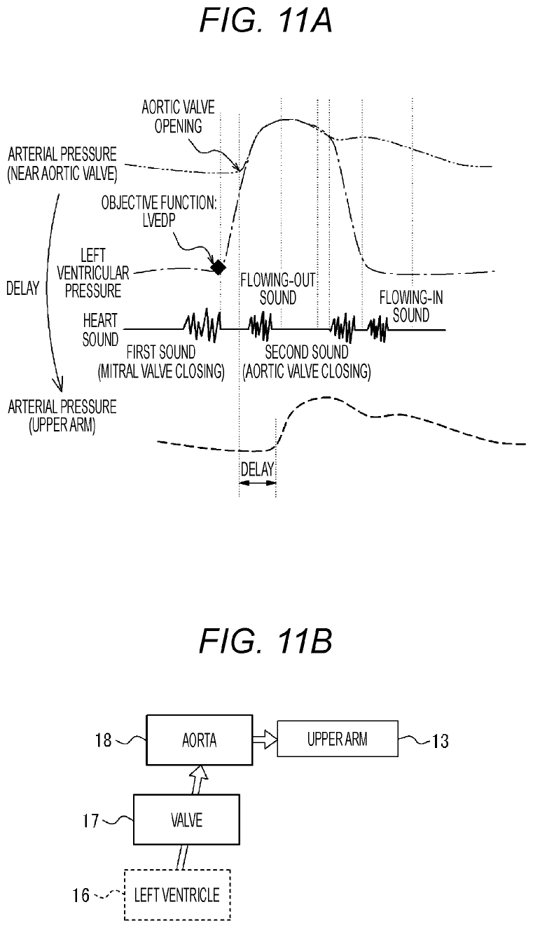

As illustrated in FIGS. 10A and 10B, as the pressure in a left ventricle 16 rises, an aortic valve 17 opens, and blood flows into an aorta 18. As illustrated in FIGS. 11A and 11B, the blood flow passing through the aortic valve 17 reaches an arm 13 of a patient, which is an upper portion of the arm, with a delay. The arterial-pressure waveform of the arm 13 is a curve that is the arterial-pressure waveform near the aortic valve 17 shifted by the delay.

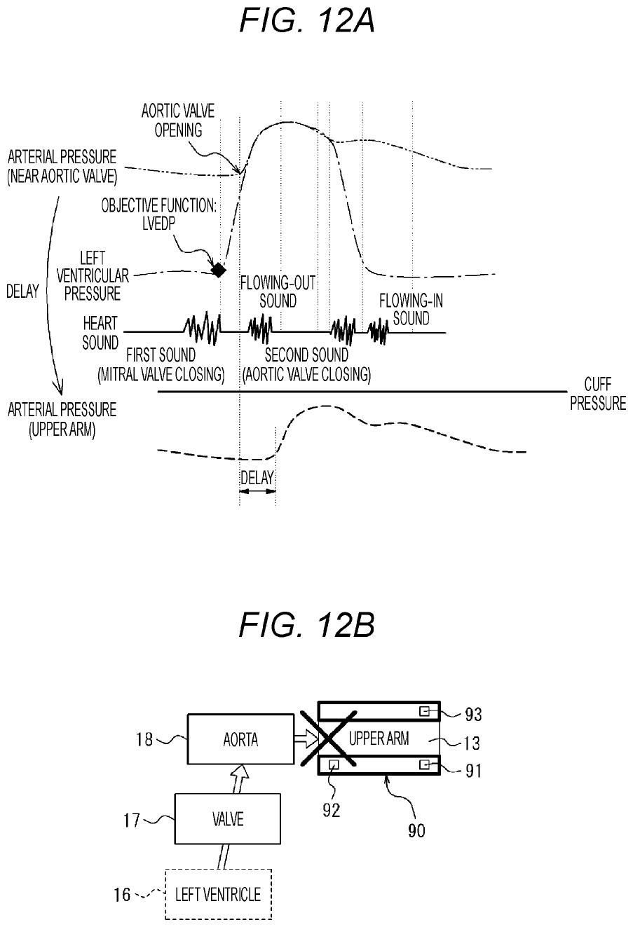

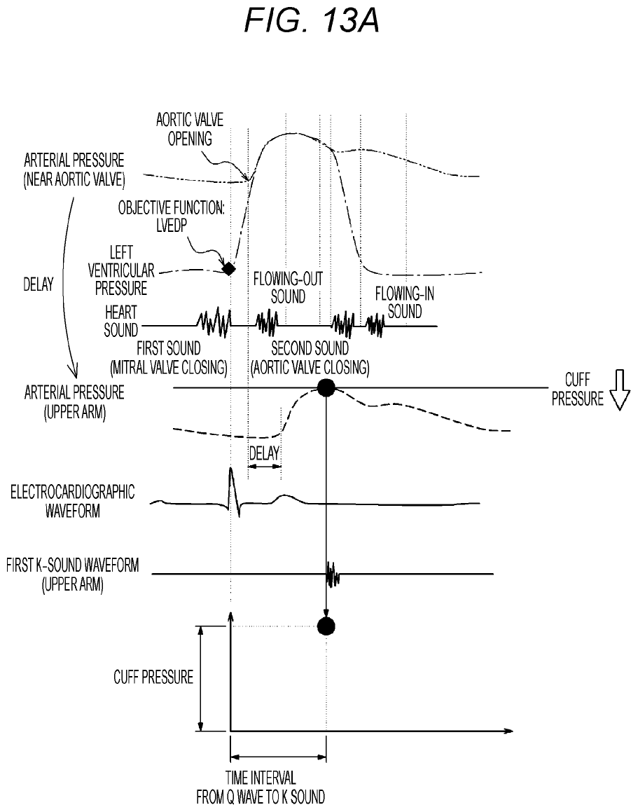

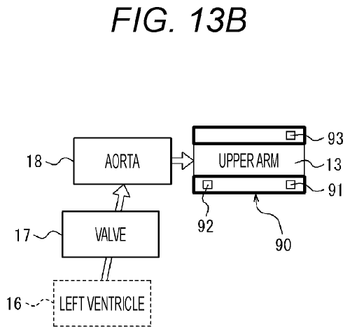

As illustrated in FIGS. 12A and 12B, when a cuff pressure is relatively high, blood does not flow to the arm 13. The cuff pressure is the pressure of a cuff 90 attached to (or wrapped around) the arm 13 of the patient. The cuff 90 contains a first K-sound microphone 91, a second K-sound microphone 92, and a pressure sensor 93. As illustrated in FIGS. 13A and 13B, when the cuff pressure is lowered while waiting for a K sound while taking the electrocardiographic waveform, the blood starts to flow to the arm 13. When a K sound generated by the blood flowing is captured by the first K-sound microphone 91, a time interval from a Q wave to the K sound, and a cuff pressure measured by the pressure sensor 93 are recorded. Instead of the time interval from a Q wave to the K sound, a time interval from a heart sound to the K sound may be recorded. The distance between the first K-sound microphone 91 and the second K-sound microphone 92 is known, and can be, for example, 3 cm to 5 cm. The delay between the aortic valve 17 and the first K-sound microphone 91 is calculated according to the difference between a timing at which a signal enters the first K-sound microphone 91 and a timing at which the signal enters the second K-sound microphone 92, and the distance between the aortic valve 17 and the first K-sound microphone 91.

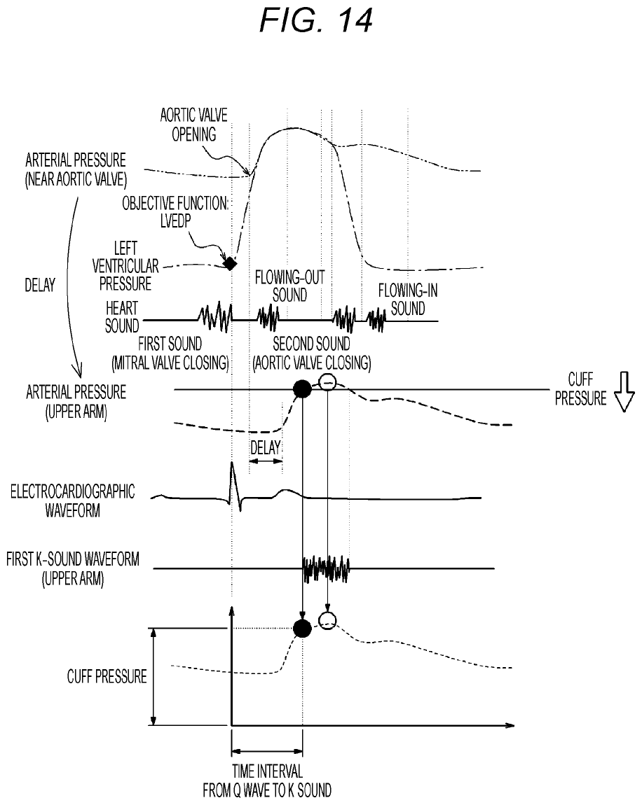

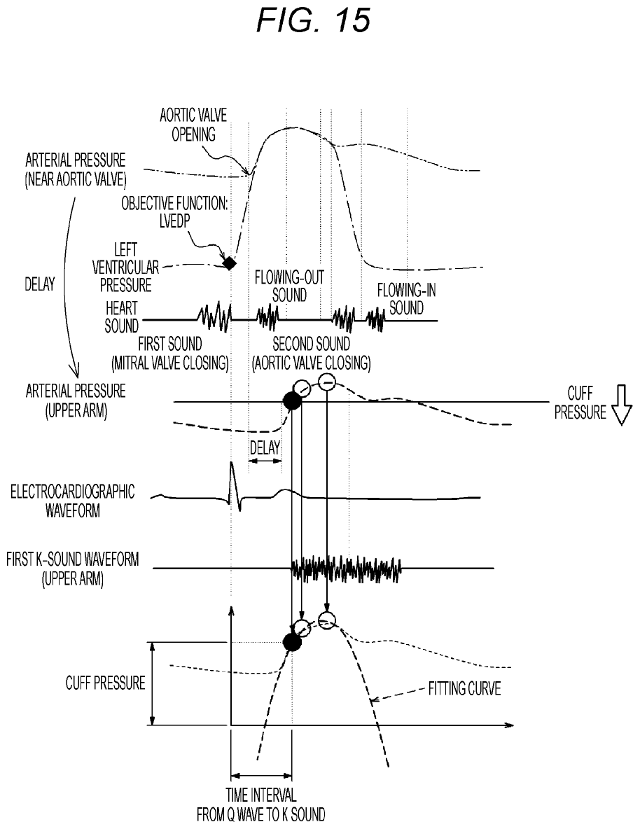

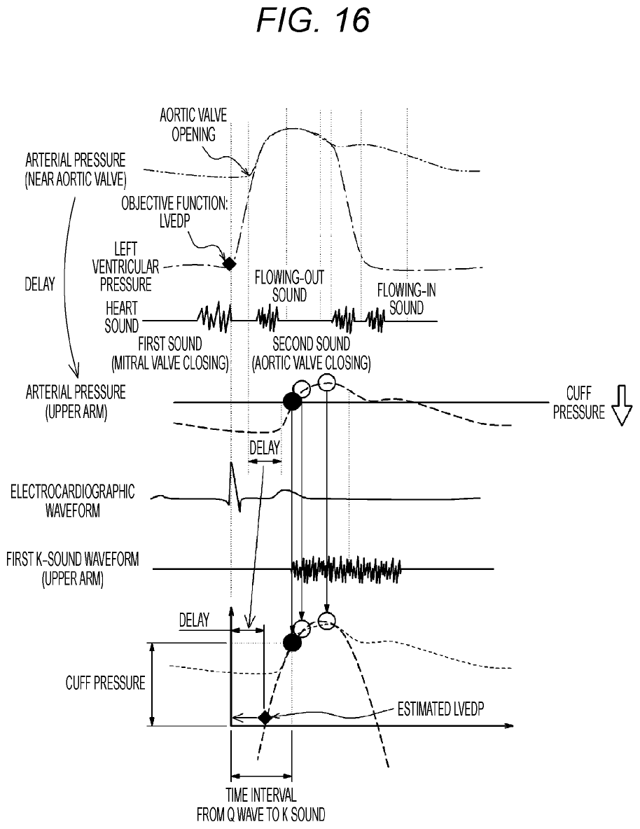

As illustrated in FIG. 14, when the cuff pressure is further lowered, start timings of K sounds in the next and subsequent heartbeats become earlier. When the K sound is captured by the first K-sound microphone 91 for each heartbeat, the time interval from a Q wave to the K sound, and a cuff pressure measured by the pressure sensor 93 are recorded. Instead of the time interval from a Q wave to the K sound, a time interval from a heart sound to the K sound may be recorded. As illustrated in FIG. 15, a curve, such as a polynomial curve or a cosine curve, can be made to fit the record of the time interval and the cuff pressure. Ideally, this curve has the same waveform as the left-ventricular-pressure waveform, however, it is difficult to fit the waveform of the curve to the left-ventricular-pressure waveform. As illustrated in FIG. 16, the pressure value at a time point at which the delay elapses from the timing of a Q wave is an estimated left ventricular end-diastolic pressure (LVEDP).

Since in the comparative example, the cuff pressure is gradually lowered for each heartbeat, and the acquisition of the information regarding the arterial pressure is completed with the plurality of beats, an error in the start timing of the K sound caused by a variation of the arterial pressure between the heartbeats can affect the information. In contrast, in the present disclosure, since acquisition of information regarding an arterial pressure is completed with one beat, it is not necessary to consider such an error as in the comparative example.

Set forth below with reference to the accompanying drawings is a detailed description of embodiments of an arterial-pressure estimation apparatus, an arterial-pressure estimation system, and an arterial-pressure estimation method. Note that since embodiments described below are preferred specific examples of the present disclosure, although various technically preferable limitations are given, the scope of the present disclosure is not limited to the embodiments unless otherwise specified in the following descriptions.

In the drawings, the same or corresponding portions are denoted by the same reference numerals. In the description of the present embodiment, the description of the same or corresponding portions will be omitted or simplified as appropriate.

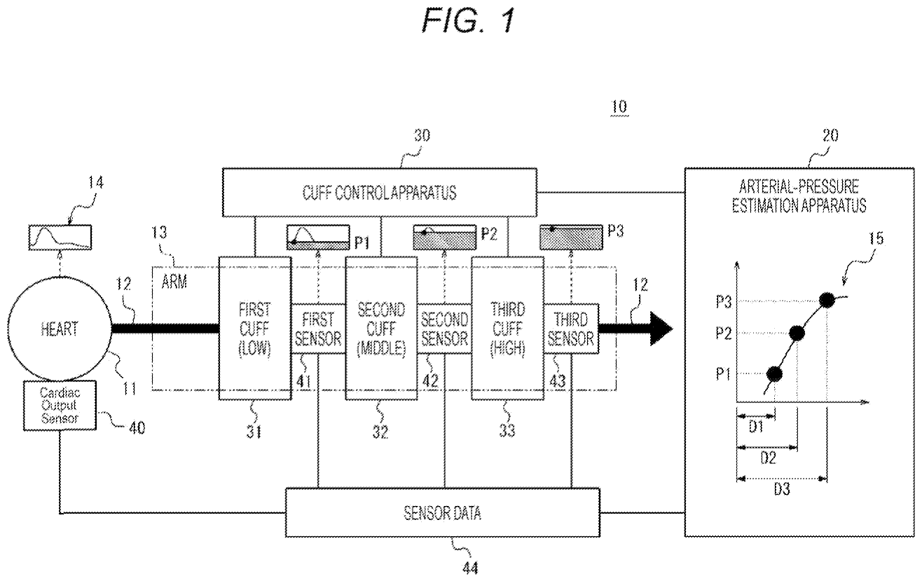

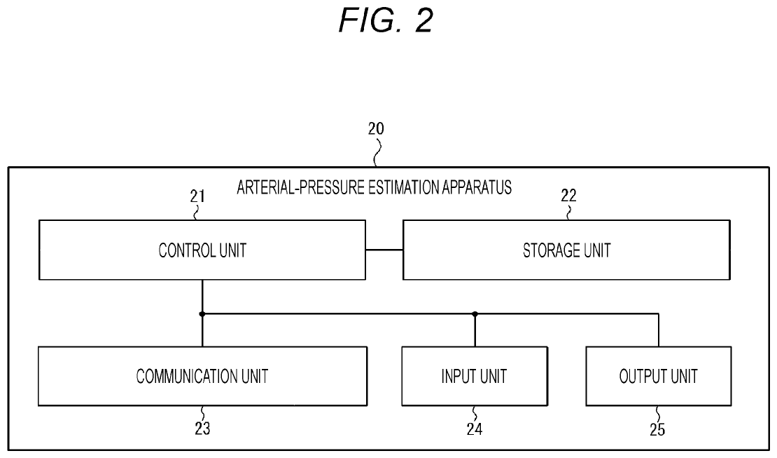

An outline of the present embodiment will be described with reference to FIGS. 1 and 2.

In an arterial-pressure estimation system 10 according to the present embodiment, two or more (i.e., at least two) inflatable units compress “two or more locations” (i.e., at least two locations) of a blood vessel 12 downstream of an aorta 18 by a higher compression at the more downstream location from the aorta (i.e., the further downstream from the aorta, the higher the compression). The two or more sensors detect blood flows generated, for one heartbeat, at the “two or more locations”. A control unit 21 acquires sensor data 44 indicating timings at which the blood flows are detected when the “two or more locations” are compressed. The control unit 21 refers to the acquired sensor data 44 to estimate a temporal variation of the arterial pressure.

According to the present embodiment, a temporal variation of an arterial pressure corresponding to one heartbeat can be estimated. Therefore, a temporal variation of an arterial pressure can be non-invasively estimated with relatively high precision.

In the present embodiment, the control unit 21 estimates an arterial-pressure waveform as a temporal variation of the arterial pressure. Therefore, an arterial-pressure waveform for one beat can be non-invasively estimated with relatively high precision.

In the present embodiment, the “two or more locations” can be “three locations”. Therefore, a temporal variation of an arterial pressure can be estimated with relatively higher precision than in a case where only “two locations” are compressed to detect blood flows only at the “two locations”. In addition, it can be easier to attach each inflatable unit and each sensor to a body part, such as the arm 13, than in a case where “four or more locations” are compressed to detect blood flows at the “four or more locations”. In the present embodiment, each of the “three locations” corresponds to a hemostasis breaking-through pressure measurement unit containing a sensor and a cuff.

A configuration of the arterial-pressure estimation system 10 according to the present embodiment will be described with reference to FIG. 1.

The arterial-pressure estimation system 10 can include an arterial-pressure estimation apparatus 20, a cuff control apparatus 30, a first cuff 31, a second cuff 32, a third cuff 33, a cardiac output sensor 40, a first sensor 41, a second sensor 42, and a third sensor 43.

The arterial-pressure estimation apparatus 20 can be a computer. The arterial-pressure estimation apparatus 20 can be, for example, dedicated equipment, general-purpose equipment, such as a personal computer (PC), or server equipment belonging to a cloud computing system or another computing system.

The cuff control apparatus 30 is equipment that controls the first cuff 31, the second cuff 32, and the third cuff 33. The cuff control apparatus 30 controls the first cuff 31, the second cuff 32, and the third cuff 33, so that the cuff pressures are increased stepwise from the upstream side of the heart of the patient, that is, from the side closer to a heart 11. The cuff pressures may be set by any method, but in the present embodiment, the first cuff 31, the second cuff 32, and the third cuff 33 are set within the range of the lowest blood pressure and the highest blood pressure measured with a sphygmomanometer such that the pressure P1 of the first cuff 31 is set to a lower blood pressure than the pressure P3 of the third cuff 33. For example, a pressure P1 of the first cuff 31 can be set to a value slightly higher than the lowest blood pressure, for example, by 5 mmHg. A pressure P3 of the third cuff 33 can be set to a value slightly lower than the highest blood pressure, for example, lower by 5 mmHg. A pressure P2 of the second cuff 32 is set to an intermediate value between the pressure P1 and the pressure P3. The cuff control apparatus 30 communicates with the arterial-pressure estimation apparatus 20 directly or via a network, such as a local area network (LAN) or the Internet.

The three inflatable units of the first cuff 31, the second cuff 32, and the third cuff 33 correspond to the “three locations” on a one-to-one basis, and compress the corresponding locations. In the present embodiment, the first cuff 31 is attached to the most upstream side (i.e., closest to the aorta of the heart) of the arm 13, the third cuff 33 is attached to the most downstream side (i.e., furthest away from aorta of the heart) of the arm 13, and the second cuff 32 is attached between the first cuff 31 and the third cuff 33. In the present embodiment, the distances between the cuffs 31, 32, 33 are fixed so that a blood propagation time is known, but the blood propagation time may be measured every time. For example, if the distances between the first cuff 31, the second cuff 32, and the third cuff 33 are each 50 mm, and a propagation speed of the blood is 1000 mm/s, a known blood propagation time is 50 ms, but a characteristic value (i.e., blood propagation time) of an individual may be measured with the tightening pressures in all the cuffs equalized, and the characteristic value may be used. In that case, a shift may be made possible from a blood propagation time measurement mode in which all the cuffs are not tightened or the same pressure is applied to all the cuffs, to an arterial-pressure waveform measurement mode in which the respective pressures for the cuffs according to the present embodiment are applied. As a modification of the present embodiment, each of the three inflatable units may be, for example, an airbag (or bladder), instead of the cuff. The three airbags can be three inflatable units that may be contained in a common cuff.

The cardiac output sensor 40 is a sensor that detects the cardiac output of the heart 11. The cardiac output sensor 40 is an electrocardiogram (ECG) sensor in the present embodiment, but may be a sound sensor that detects a closing sound of a mitral valve. The cardiac output sensor 40 communicates with the arterial-pressure estimation apparatus 20 directly or via a network, such as a LAN or the Internet.

The three sensors of the first sensor 41, the second sensor 42, and the third sensor 43 correspond to the “three locations” on a one-to-one basis, and detect blood flows generated at the corresponding locations. The first sensor 41 is disposed downstream of the first cuff 31 and upstream of the second cuff 32, and detects a blood flow generated downstream of the first cuff 31. The second sensor 42 is disposed downstream of the second cuff 32 and upstream of the third cuff 33, and detects a blood flow generated downstream of the second cuff 32. The third sensor 43 is disposed downstream of the third cuff 33 and detects a blood flow generated downstream of the third cuff 33. In the present embodiment, each sensor 41, 42, 43, can be a sound sensor that detects a sound generated by blood flowing. However, each sensor 41, 42, 43 may be a photoplethysmogram (PPG) sensor or an ultrasonic sensor that measures a blood flow by an ultrasonic Doppler method. The first sensor 41, the second sensor 42, and the third sensor 43 communicate with the arterial-pressure estimation apparatus 20 directly or via a network, such as a LAN or the Internet. As a modification of the present embodiment, the three sensors 41, 42, 43 may be each integrated with the corresponding cuff (i.e., integrated within a single cuff).

The arterial-pressure estimation apparatus 20 acquires, as the sensor data 44, signals output from the cardiac output sensor 40, the first sensor 41, the second sensor 42, and the third sensor 43. The sensor data 44 is data indicating a cardiac output timing T0, a first detection timing T1, a second detection timing T2, and a third detection timing T3. The cardiac output timing T0 is a timing at which a cardiac output is detected by the cardiac output sensor 40. The cardiac output timing T0 is treated as a reference timing of the heart 11. The reference timing is a timing that can be identified for each heartbeat. The first detection timing T1 is a timing at which a blood flow is detected by the first sensor 41. The second detection timing T2 is a timing at which a blood flow is detected by the second sensor 42. The third detection timing T3 is a timing at which a blood flow is detected by the third sensor 43.

The arterial-pressure estimation apparatus 20 refers to the sensor data 44 to calculate a time difference D1 from the cardiac output timing T0 to the first detection timing T1. The arterial-pressure estimation apparatus 20 refers to the sensor data 44 to calculate a time difference D2 obtained by subtracting a blood propagation time between the first cuff 31 and the second cuff 32, from a time difference from the cardiac output timing T0 to the second detection timing T2. The arterial-pressure estimation apparatus 20 refers to the sensor data 44 to calculate a time difference D3 obtained by subtracting a blood propagation time between the first cuff 31 and the third cuff 33, from a time difference from the cardiac output timing T0 to the third detection timing T3.

The arterial-pressure estimation apparatus 20 estimates an arterial-pressure waveform 15 of one beat, from the time difference D1, the time difference D2, and the time difference D3, and the corresponding pressure P1 of the first cuff 31, pressure P2 of the second cuff 32, and pressure P3 of the third cuff 33. This arterial-pressure waveform 15 corresponds to an arterial-pressure waveform 14 of the aorta 18. Therefore, the arterial-pressure estimation apparatus 20 can estimate an LVEDP from an estimated arterial-pressure waveform 14.

According to the present embodiment, an LVEDP value estimated by the arterial-pressure estimation apparatus 20 can be utilized to provide a determination index in a medical examination and treatment for identifying heart failure of a patient. On the basis of determination index, it is possible to recommend changing the prescription of a diuretic or the like for the patient, determine or recommend hospitalization of the patient, or determine or recommend discharge of the patient from the hospital.

A configuration of the arterial-pressure estimation apparatus 20 according to the present embodiment will be described with reference to FIG. 2.

The arterial-pressure estimation apparatus 20 can include the control unit 21, a storage unit 22, a communication unit 23, an input unit 24, and an output unit 25.

The control unit 21 can include at least one processor, at least one programmable circuit, at least one dedicated circuit, or any combination of the at least one processor, the at least one programmable circuit, and the at least one dedicated circuit. The processor can be a general-purpose processor, such as a central processing unit (CPU) or a graphics processing unit (GPU), or a dedicated processor specialized for specific processing. The programmable circuit can be, for example, a field-programmable gate array (FPGA). The dedicated circuit can be, for example, an application specific integrated circuit (ASIC). The control unit 21 executes processing related to operations of the arterial-pressure estimation apparatus 20 while controlling each unit of the arterial-pressure estimation apparatus 20.

The storage unit 22 can include at least one semiconductor memory, at least one magnetic memory, at least one optical memory, or any combination of the at least one semiconductor memory, the at least one magnetic memory, and the at least one optical memory. The semiconductor memory can be, for example, a random access memory (RAM) or a read only memory (ROM). The RAM can be, for example, a static random access memory (SRAM) or a dynamic random access memory (DRAM). The ROM can be, for example, an electrically erasable programmable read only memory (EEPROM). The storage unit 22 can function as, for example, a main storage, an auxiliary storage, or a cache memory. The storage unit 22 stores data used for operations of the arterial-pressure estimation apparatus 20, and data obtained by operations of the arterial-pressure estimation apparatus 20.

The communication unit 23 can include at least one communication interface. The communication interface can be, for example, a LAN interface, an interface compatible with a mobile communication standard, such as long term evolution (LTE), the 4^{th} Generation (4G) standard, or the 5^{th} Generation (5G) standard, or an interface compatible with near-field communication, such as Bluetooth (registered trademark). The communication unit 23 receives data used for operations of the arterial-pressure estimation apparatus 20, and transmits data obtained by operations of the arterial-pressure estimation apparatus 20.

The input unit 24 can include at least one input interface. The input interface can be, for example, physical keys, capacitive keys, a pointing device, a touchscreen provided integrally with a display, an imaging apparatus, such as a camera, or a microphone. The input unit 24 receives an operation of inputting data used for operations of the arterial-pressure estimation apparatus 20. The input unit 24 may not be provided for the arterial-pressure estimation apparatus 20, but may be connected, as external input equipment, to the arterial-pressure estimation apparatus 20. As the connection method, for example, any scheme, such as Universal Serial Bus (USB), High-Definition Multimedia Interface (HDMI®), or Bluetooth®, may be used.

The output unit 25 can include at least one output interface. The output interface can be, for example, a display or a speaker. The display can be, for example, a liquid crystal display (LCD) or an organic electro luminescence (EL) display. The output unit 25 outputs data obtained by operations of the arterial-pressure estimation apparatus 20. The output unit 25 may not be provided for the arterial-pressure estimation apparatus 20, but may be connected, as external output equipment, to the arterial-pressure estimation apparatus 20. As the connection method, for example, any scheme, such as USB, HDMI®, or Bluetooth®, may be used.

Functions of the arterial-pressure estimation apparatus 20 can be implemented by a processor, as the control unit 21, executing programs according to the present embodiment. That is, the functions of the arterial-pressure estimation apparatus 20 can be implemented by software. The programs executed according to the present embodiment can enable a computer to execute operations of the arterial-pressure estimation apparatus 20 to enable the computer to function as the arterial-pressure estimation apparatus 20. That is, the computer executes operations of the arterial-pressure estimation apparatus 20 according to the programs to function as the arterial-pressure estimation apparatus 20.

The programs may be stored on a non-transitory computer-readable medium. The non-transitory computer-readable medium can be, for example, a flash memory, a magnetic recording device, an optical disc, a magneto-optical recording medium, or a ROM. The distribution of the programs is performed, for example, by selling, transferring, or lending a portable medium, such as Secure Digital (SD) card, a digital versatile disc (DVD), or a compact disc read only memory (CD-ROM), storing the programs. The programs may be distributed by storing the programs in a storage of a server and transferring the programs from the server to another computer. The programs may be provided as a program product.

For example, a computer can temporarily store, in the main storage, the programs stored in a portable medium or the programs transferred from a server. Then, the computer can read, with a processor, the programs stored in the main storage, and executes, with the processor, processing according to the read programs. A computer may read the programs directly from a portable medium, and execute processing according to the programs. Each time the program is transferred from a server to a computer, the computer may sequentially execute processing according to the received program. The programs may not be transferred from a server to a computer, but the processing may be executed by what is called an application service provider (ASP) service in which the functions are implemented only by execution instructions and result acquisition. The programs include information that is used for processing by a computer and is equivalent to the programs. For example, data that is not a direct command to a computer but has a property that defines processing of a computer corresponds to “data equivalent to the programs”.

Part or all of the functions of the arterial-pressure estimation apparatus 20 may be implemented by a programmable circuit or a dedicated circuit, as the control unit 21. That is, part or all of the functions of the arterial-pressure estimation apparatus 20 may be implemented by hardware.

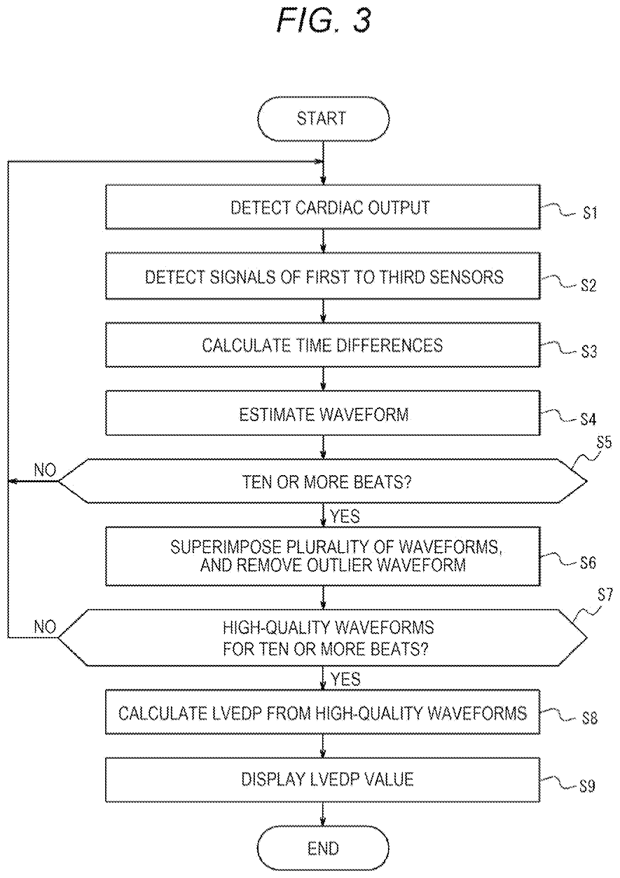

Operations of the arterial-pressure estimation system 10 according to the present embodiment will be described with reference to FIG. 3. These operations correspond to an arterial-pressure estimation method according to the present embodiment.

In S1, the cardiac output sensor 40 detects a cardiac output of the heart 11. Specifically, the cardiac output sensor 40 measures an electrocardiographic waveform. The cardiac output sensor 40 outputs a signal indicating the measured electrocardiographic waveform.

In S2, the first sensor 41, the second sensor 42, and the third sensor 43 detect blood flows generated, for one heartbeat, at “three locations” of the blood vessel 12 downstream of the aorta 18 when the “three locations” are compressed by higher pressures at the more downstream locations (i.e., the further downstream the location (or site) is, the higher the compression). Specifically, the first sensor 41 detects a K sound generated by the blood flow breaking through the first cuff 31. The first sensor 41 outputs a signal indicating the waveform of the K sound. The second sensor 42 detects a K sound generated by the blood flow breaking through the second cuff 32. The second sensor 42 outputs a signal indicating the waveform of the K sound. The third sensor 43 detects a K sound generated by the blood flow breaking through the third cuff 33. The third sensor 43 outputs a signal indicating the waveform of the K sound.

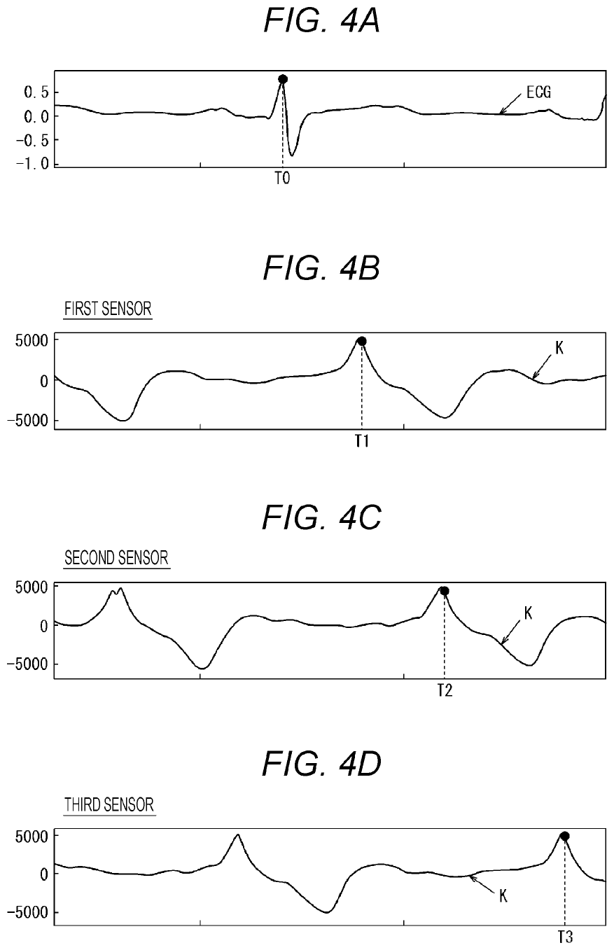

In S3, the control unit 21 of the arterial-pressure estimation apparatus 20 acquires sensor data 44. The sensor data 44 includes data indicating the timing at which the cardiac output has been detected in S1. Specifically, the sensor data 44 includes, as such data, data indicating the electrocardiographic waveform measured by the cardiac output sensor 40. As illustrated in FIG. 4A, from the electrocardiographic waveform measured by the cardiac output sensor 40, the control unit 21 identifies a cardiac output timing T0. The sensor data 44 further includes data indicating the timings at which the blood flows generated, for the one heartbeat, at the “three locations” have been detected in step S2. Specifically, the sensor data 44 includes, as such data, data indicating the waveforms of the K sounds detected by the first sensor 41, the second sensor 42, and the third sensor 43. As illustrated in FIG. 4B, from the waveform of the K sound detected by the first sensor 41, the control unit 21 identifies, as a first detection timing T1, the timing at which the blood has broken through the first cuff 31. As illustrated in FIG. 4C, from the waveform of the K sound detected by the second sensor 42, the control unit 21 identifies, as a second detection timing T2, the timing at which the blood has broken through the second cuff 32. As illustrated in FIG. 4D, from the waveform of the K sound detected by the third sensor 43, the control unit 21 identifies, as a third detection timing T3, the timing at which the blood has broken through the third cuff 33. Between the first detection timing T1 and the second detection timing T2, there is a time difference due to a delay in the breaking-through of the blood because of the higher compression pressure on the downstream side, and a delay because of the second cuff 32 being downstream. Between the second detection timing T2 and the third detection timing T3, there is also a time difference due to a delay in the breaking-through of the blood because of the higher compression pressure on the downstream side, and a delay because of the third cuff 33 being downstream.

According to the distances between the “three locations”, the control unit 21 of the arterial-pressure estimation apparatus 20 corrects the timings indicated by the acquired sensor data 44. Specifically, the control unit 21 corrects the timings by subtracting delay times corresponding to the distances between the “three locations”, from the timings indicated by the sensor data 44. More specifically, the control unit 21 calculates, as a time difference D1, the time difference from the cardiac output timing T0 to the first detection timing T1. The control unit 21 calculates a time difference D2 by subtracting a delay time corresponding to the distance between the first cuff 31 and the second cuff 32, from the time difference from the cardiac output timing T0 to the second detection timing T2. The control unit 21 calculates a time difference D3 by subtracting a delay time corresponding to the distance between the first cuff 31 and the third cuff 33, from the time difference from the cardiac output timing T0 to the third detection timing T3.

The cardiac output timing T0 may be identified from a heart sound instead of the electrocardiographic waveform. In this case, in S1, the cardiac output sensor 40 can detect a heart sound including a closing sound of a mitral valve. The cardiac output sensor 40 outputs a signal indicating the detected heart sound. In S3, sensor data 44 includes data indicating the heart sound detected by the cardiac output sensor 40. From the heart sound detected by the cardiac output sensor 40, the control unit 21 of the arterial-pressure estimation apparatus 20 can identify a cardiac output timing T0.

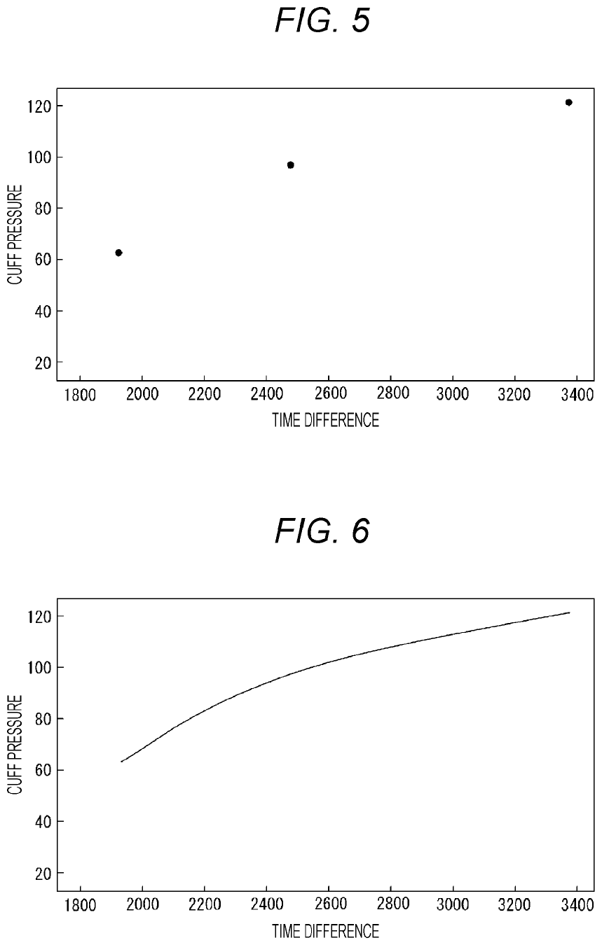

In S4, the control unit 21 of the arterial-pressure estimation apparatus 20 refers to the sensor data 44 acquired in S3 to estimate a temporal variation of the arterial pressure. Specifically, the control unit 21 estimates a temporal variation of the arterial pressure according to the timings corrected in S3 and the pressures by which the “three locations” are compressed. More specifically, as illustrated in FIG. 5, the control unit 21 can plot the time difference D1, the time difference D2, and the time difference D3 calculated in S3, and the corresponding pressure P1 of the first cuff 31, pressure P2 of the second cuff 32, and pressure P3 of the third cuff 33. As illustrated in FIG. 6, the control unit 21 estimates a curve of an arterial-pressure waveform by performing a spline interpolation on the plotted three points.

In S5, the control unit 21 of the arterial-pressure estimation apparatus 20 determines whether or not estimation results of arterial-pressure waveforms, for example, for ten or more heartbeats have been obtained. If estimation results of arterial-pressure waveforms, for example, for ten or more heartbeats have not been obtained, the processing from S1 to S4 is performed again. If estimation results of arterial-pressure waveforms, for example, for ten or more heartbeats have been obtained, the processing of S6 is performed.

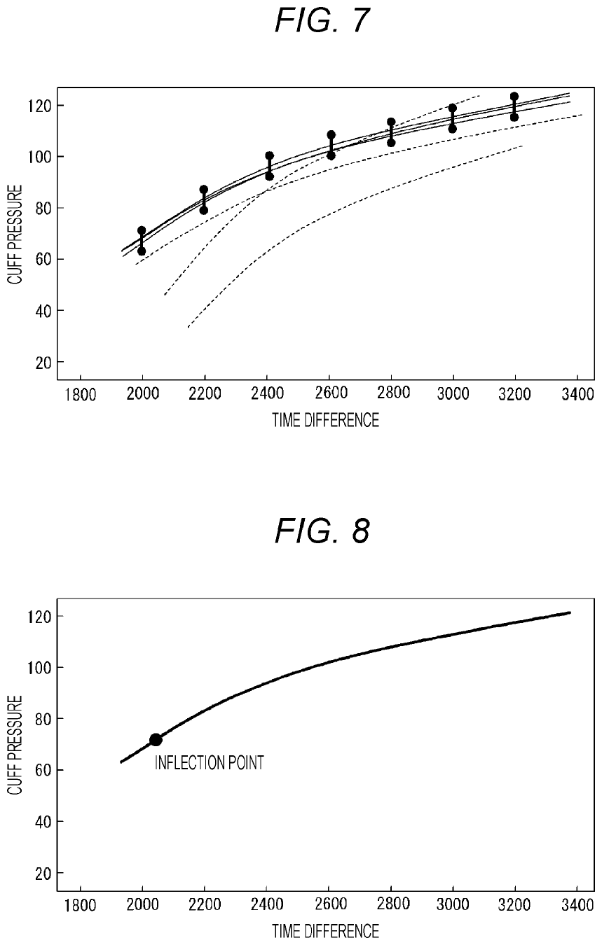

In S6, the control unit 21 of the arterial-pressure estimation apparatus 20 executes statistical processing on the sensor data 44 acquired, for example, for the ten or more heartbeats. Specifically, as illustrated in FIG. 7, the control unit 21 superimposes the curves of the plurality of arterial-pressure waveforms estimated, for example, for the ten or more heartbeats. The control unit 21 can calculate an average of the curves of the plurality of arterial-pressure waveforms, and can remove, as an outlier, the curves that are not within a certain distance from the average. The remaining curves that are not removed can be considered to be high-quality-waveform curves.

In S7, the control unit 21 of the arterial-pressure estimation apparatus 20 determines whether or not high-quality-waveform curves, for example, for ten or more heartbeats have been obtained. If high-quality-waveform curves, for example, for ten or more heartbeats have not been obtained, the processing from S1 to S6 is performed again. If high-quality-waveform curves, for example, for ten or more heartbeats have been obtained, the processing of S8 is performed.

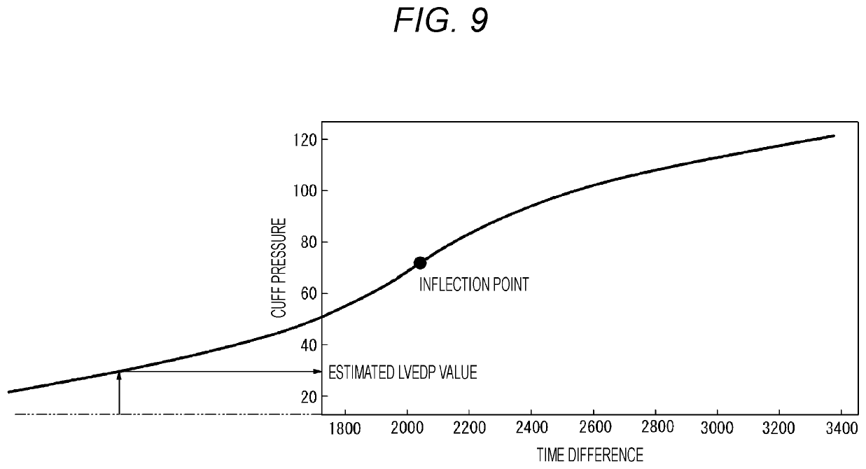

In S8, the control unit 21 of the arterial-pressure estimation apparatus 20 estimates an LVEDP on the basis of an estimation result of a temporal variation of the arterial pressure. Specifically, the control unit 21 estimates a left-ventricular-pressure waveform according to an estimated arterial-pressure waveform. From the estimated left-ventricular-pressure waveform, the control unit 21 acquires an estimated value of an LVEDP. In the present embodiment, the control unit 21 estimates a temporal variation of the arterial pressure on the basis of a result of the statistical processing executed in S6. More specifically, as illustrated in FIG. 8, the control unit 21 averages the high-quality-waveform curves obtained in S6. The control unit 21 identifies an inflection point of the averaged curve. As illustrated in FIG. 9, the control unit 21 adds a curve beyond the inflection point by extrapolating a line obtained by rotating the averaged curve by 180 degrees. As a result, a curve of a left-ventricular-pressure waveform is obtained. The control unit 21 can acquire, as an estimated value of an LVEDP, a pressure value at the time point when a delay time corresponding to the distance between the aortic valve 17 and the uppermost location among the “three locations” has elapsed from a reference timing identified for each heartbeat. Specifically, as illustrated in FIG. 9, the control unit 21 can identify, as an estimated LVEDP value, a cuff pressure corresponding to a time difference corresponding to a blood propagation time corresponding to the distance between the aortic valve 17 and the first cuff 31, from the cardiac output timing T0. The distance between the aortic valve 17 and the first cuff 31 may be predetermined as a fixed value, may be measured using computed tomography (CT) or magnetic resonance imaging (MRI), or may be calculated according to the path length of a catheter at a time of medical care. The blood propagation time between the aortic valve 17 and the first cuff 31 may be calculated from the blood propagation time between the first cuff 31, the second cuff 32, and the third cuff 33. For example, if the distance between the aortic valve 17 and the first cuff 31 is 150 mm, and the distance between the first cuff 31 and the second cuff 32 is 50 mm, when the blood propagation time between the first cuff 31 and the second cuff 32 is 50 millisecond (ms), the blood propagation time between the aortic valve 17 and the first cuff 31 is 150 ms. The blood propagation time may be considered in terms of each sensor instead of each cuff.

In S9, the control unit 21 of the arterial-pressure estimation apparatus 20 outputs the estimated value of an LVEDP acquired in S8. For example, the control unit 21 displays the estimated value of an LVEDP on a display as the output unit 25. Alternatively, the control unit 21 outputs the estimated value of an LVEDP by sound from a speaker as the output unit 25. Alternatively, the control unit 21 makes the communication unit 23 transmit the estimated value of an LVEDP. The communication unit 23 transmits the estimated value of an LVEDP to another apparatus directly or via a network, such as a LAN or the Internet.

As described above, in the present embodiment, the control unit 21 of the arterial-pressure estimation apparatus 20 acquires sensor data 44 indicating the timings at which blood flows generated and detected, for one heartbeat, at “three locations” of the blood vessel 12 downstream of the aorta 18 when the “three locations” are compressed by higher pressures at the more downstream locations. The control unit 21 refers to the acquired sensor data 44 to estimate a temporal variation of the arterial pressure.

According to the present embodiment, a temporal variation of an arterial pressure corresponding to one heartbeat can be estimated. Therefore, a temporal variation of an arterial pressure can be non-invasively estimated with relatively high precision.

As a modification of the present embodiment, in S8, instead of extrapolating a line obtained by rotating an averaged curve by 180 degrees, the control unit 21 of the arterial-pressure estimation apparatus 20 may generate a curve, for example, a polynomial curve or a cosine curve similar to the polynomial curve or the cosine curve in the comparative example that may be added as a curve beyond the inflection point, and which fits the averaged curve of a polynomial or a cosine curve. Alternatively, the control unit 21 may make a preliminarily measured arterial-pressure waveform of a subject fit. A curve suitable for the subject can be made to fit, so that a curve of a left-ventricular-pressure waveform can be obtained.

As a modification of the present embodiment, the control unit 21 of the arterial-pressure estimation apparatus 20 may acquire sensor data 44 for a plurality of times, for example, which is different from ten, of heartbeats, and refer to the acquired sensor data 44 to estimate a temporal variation of the arterial pressure. Alternatively, the control unit 21 may acquire sensor data 44 for only one heartbeat, and refer to the acquired sensor data 44 to estimate a temporal variation of the arterial pressure. In this case, the control unit 21 may not estimate the curve of the arterial-pressure waveform but may input data corresponding to the three points illustrated in FIG. 5, into a trained model, and use the trained model to estimate an LVEDP. That is, the control unit 21 may input an estimation result of a temporal variation of the arterial pressure, into a trained model, and acquire an estimated value of an LVEDP, from the trained model.

The present disclosure is not limited to the above-described embodiment. For example, two or more blocks described in the block diagrams may be integrated, or one block may be divided. Instead of executing two or more steps described in the flowchart in chronological order according to the description, the steps may be executed in parallel or in a different order, depending on the processing capability of an apparatus that executes each step, or as needed. In addition, modifications can be made without departing from the gist of the present disclosure.

The detailed description above describes embodiments of an arterial-pressure estimation apparatus, an arterial-pressure estimation system, and an arterial-pressure estimation method. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. An arterial-pressure estimation apparatus comprising:
a control unit configured to:
acquire sensor data indicating timings at which blood flows generated, for one heartbeat, at two or more locations of a blood vessel downstream of an aorta when the two or more locations are compressed such that a location of the two or more locations that is further downstream of the aorta has a higher compression pressure than a location of the two more locations that is further upstream of the aorta; and
estimate a temporal variation of an arterial pressure based on the acquired sensor data.

2. The arterial-pressure estimation apparatus according to claim 1, wherein the control unit is configured to:
correct, according to a distance between the two or more locations, the timings indicated by the acquired sensor data; and
estimate, according to the corrected timings, and pressures by which the two or more locations are compressed, a temporal variation of the arterial pressure.

3. The arterial-pressure estimation apparatus according to claim 2, wherein the control unit is configured to:
correct the timings by subtracting a delay time corresponding to the distance, from the timings indicated by the acquired sensor data.

4. The arterial-pressure estimation apparatus according to claim 1, wherein the control unit is configured to:
estimate a left ventricular end-diastolic pressure on a basis of an estimation result of the temporal variation of the arterial pressure.

5. The arterial-pressure estimation apparatus according to claim 4, wherein the control unit is configured to:
estimate, as the temporal variation of the arterial pressure, an arterial-pressure waveform;
estimate, according to the estimated arterial-pressure waveform, a left-ventricular-pressure waveform; and
acquire, from the estimated left-ventricular-pressure waveform, an estimated value of the left ventricular end-diastolic pressure.

6. The arterial-pressure estimation apparatus according to claim 4, wherein the control unit is configured to:
input the estimation result of the temporal variation of the arterial pressure, into a trained model; and
acquire an estimated value of the left ventricular end-diastolic pressure from the trained model.

7. The arterial-pressure estimation apparatus according to claim 4, wherein the control unit is configured to:
output an estimated value of the left ventricular end-diastolic pressure.

8. The arterial-pressure estimation apparatus according to claim 1, wherein the control unit is configured to:
acquire sensor data for a plurality of heartbeats;
execute statistical processing on the sensor data for the plurality of heartbeats; and
estimate, on a basis of a result of the statistical processing, a temporal variation of the arterial pressure.

9. The arterial-pressure estimation apparatus according to claim 1, wherein the two or more locations are three locations.

10. An arterial-pressure estimation system comprising:
an arterial-pressure estimation apparatus that includes a control unit configured to acquire sensor data indicating timings at which blood flows generated, for one heartbeat, at two or more locations of a blood vessel downstream of an aorta when the two or more locations are compressed such that a location of the two or more locations that is further downstream of the aorta has a higher compression pressure than a location of the two more locations that is further upstream of the aorta, and estimate a temporal variation of an arterial pressure based on the acquired sensor data; and
two or more sensors corresponding to the two or more locations on a one-to-one basis, and configured to detect blood flows for the one heartbeat generated at the corresponding locations, respectively.

11. The arterial-pressure estimation system according to claim 10, further comprising:
two or more inflatable units corresponding to the two or more locations on a one-to-one basis, and configured to compress the corresponding locations, respectively.

12. The arterial-pressure estimation system according to claim 11, wherein the two or more inflatable units are each a cuff.

13. The arterial-pressure estimation system according to claim 11, wherein the two or more inflatable units are each an airbag, and each of the airbags is contained in a common cuff.

14. An arterial-pressure estimation method comprising:
compressing two or more locations of a blood vessel downstream of an aorta with two or more inflatable units, and wherein a location of the two or more locations that is further downstream of the aorta has a higher compression pressure than a location of the two more locations that is further upstream of the aorta;
detecting blood flows generated, for one heartbeat, at the two or more locations with two or more sensors;
acquiring sensor data indicating timings at which the blood flows for the one heartbeat are detected when the two or more locations are compressed; and
estimating a temporal variation of an arterial pressure based on the acquired sensor data.

15. The arterial-pressure estimation method according to claim 14, further comprising:
correcting, according to a distance between the two or more locations, the timings indicated by the acquired sensor data; and
estimating, according to the corrected timings, and pressures by which the two or more locations are compressed, a temporal variation of the arterial pressure.

16. The arterial-pressure estimation method according to claim 15, further comprising:
correcting the timings by subtracting a delay time corresponding to the distance, from the timings indicated by the acquired sensor data.

17. The arterial-pressure estimation method according to claim 14, further comprising:
estimating a left ventricular end-diastolic pressure on a basis of an estimation result of the temporal variation of the arterial pressure.

18. The arterial-pressure estimation method according to claim 17, further comprising:
estimating, as the temporal variation of the arterial pressure, an arterial-pressure waveform;
estimating, according to the estimated arterial-pressure waveform, a left-ventricular-pressure waveform; and
acquiring, from the estimated left-ventricular-pressure waveform, an estimated value of the left ventricular end-diastolic pressure.

19. The arterial-pressure estimation method according to claim 17, further comprising:
inputting the estimation result of the temporal variation of the arterial pressure, into a trained model; and
acquiring an estimated value of the left ventricular end-diastolic pressure from the trained model.

20. The arterial-pressure estimation method according to claim 14, further comprising:
acquiring sensor data for a plurality of heartbeats;
executing statistical processing on the sensor data for the plurality of heartbeats; and
estimating, on a basis of a result of the statistical processing, a temporal variation of the arterial pressure.
